(19) United States
(12) Patent Application Publication
(10) Pub. No.: US 2026/0069160 A1
(43) Pub. Date: Mar. 12, 2026

(54) Body Part Cushion

(71) Applicant: Siemens Healthineers AG, Forchheim (DE)

(72) Inventors: Vincent Kelber, Bamberg (DE); Volker Matschl, Bamberg (DE); Johann Sukkau, Herzogenaurach (DE); Hans Weber, Erlangen (DE)

(73) Assignee: Siemens Healthineers AG, Forchheim (DE)

(21) Appl. No.: 19/321,362

(22) Filed: Sep. 8, 2025

(30) Foreign Application Priority Data
Sep. 9, 2024 (EP) 24199104.1

(51) Int. Cl. A61B 5/055; A61B 5/00; A61B 5/01

(52) U.S. Cl. CPC A61B 5/055; A61B 5/01; A61B 5/70; A61B 2562/0247; A61B 2562/0261

(57) ABSTRACT

A body part cushion for supporting a body part during a magnetic resonance examination with a magnetic resonance device, wherein the body part cushion has at least one presence sensor configured to acquire presence data related to the presence of the body part and a communication apparatus configured to transmit the presence data to a receiver.

### TECHNICAL FIELD

The disclosure relates to a body part cushion, particularly a head cushion, for supporting a body part, such as a head, during a magnetic resonance examination using a magnetic resonance device. Additionally, the disclosure pertains to a magnetic resonance device and a method for detecting the presence of a body part.

### BACKGROUND

Magnetic resonance examinations of patients with magnetic resonance imaging devices are performed using magnetic resonance sequences, which comprise the emission of high-frequency pulses and gradient pulses, which generate electromagnetic changes, which also have effects on the body of the patient being examined. Predicting the specific absorption rate (SAR) arising from the measurement is therefore a known step for magnetic resonance examinations of patients. An averaged SAR in particular should thereby remain below a limit value during a specified time interval, in order to avoid harming the patient, particularly due to excessing heating. A differentiation is usually made between head SAR, whole-body SAR, and exposed partial-body SAR. In order to make this determination, it is necessary to know certain information, including: the position of the patient in the patient opening, in order to differentiate a head examination from a foot examination, for example, an orientation of the patient on the patient table (head first or feet first), and the position of the relevant body regions for the head SAR and the exposed partial-body SAR.

If a head coil is used as a local coil arrangement, the position of the head, namely in the head coil, can be inferred. Particularly in the case of an FFS (feet first supine) examination, however, the patient is generally lying in a random and thus unknown position on the patient table, and the specific examination region cannot easily be determined.

A worst-case position for the patient or corresponding body parts, at which the highest SAR values would occur, is therefore often assumed in the prior art in such a situation. Thus, a maximum SAR is assumed, which is certainly safe, but can severely restrict the quality of the examination (image quality, measurement time, number of layers). In order to improve the situation in this regard, it is necessary to know or be able to determine the position of a body part on the patient table that is exposed to the pulses and particularly in a coordinate system of the magnetic resonance device, at least in the longitudinal direction of the cylindrical patient opening. Information about a head position would thereby be particularly important in order to be able to prevent the brain tissue from overheating.

In principle, it would be conceivable to determine a body position with a 3D camera. However, such a solution is quite complex and expensive. Every magnetic resonance imaging system is not necessarily equipped with a suitable 3D camera, nor is not designed to use it in such a way. Moreover, values supplied by a 3D camera may lead to additional problems with the appropriate evaluation and interpretation of the camera data, which are difficult to resolve.

The subsequently published German patent application DE 10 2024 205 084.6 proposes a body part cushion, particularly a head cushion, for supporting a body part, particularly a head, during a magnetic resonance examination, wherein the body part cushion comprises a magnetic field sensor, particularly a 3D Hall sensor (three-dimensional Hall sensor), for acquiring the local magnetic field as position data, wherein the body part cushion comprises a communication apparatus for transmitting the position data to a receiver. However, the problem here is that the patient has not necessarily positioned the body part, particularly the head, in or on the body part cushion, and also kept it there consistently throughout the measurement. When faced with uncertainty, therefore, a worst-case scenario must again be assumed.

### SUMMARY

An object of the disclosure is thus to allow the position of body parts in a magnetic resonance device to be determined more reliably.

With a body part cushion of the type mentioned in the introduction, it is provided according to the disclosure that the body part cushion has at least one presence sensor for acquiring presence data related to the presence of the body part and a communication apparatus for transmitting the presence data to a receiver.

Particular advantages arise if the body part cushion also has a magnetic field sensor for acquiring the local magnetic field as position data, and the communication apparatus is also designed to transmit the position data to the receiver. The body part cushion, the magnetic resonance device, and the method can thus be a development in particular with respect to the subject matter described in DE 10 2024 205 084.6. In this respect, particularly with regard to magnetic field measurement for position determination, position determination with position data, and use for SAR prediction, the description there can also be applied to the present disclosure. The provision of body part cushions without magnetic field sensors is also conceivable in principle, if the presence of the body part is to be established for other reasons.

According to the disclosure, it is thus proposed to provide a presence sensor as part of a body part cushion, whose measured data describes or indicates whether there actually is a body part on the body part cushion of the type for which the body part cushion is provided. Whenever it is necessary to confirm that the body part is also actually present, particularly for another function, the presence data can thus be evaluated, for example, as the basis or trigger for the other function, to the effect that the presence of the body part is monitored. In the specific application example of SAR prediction, the reliability of the assumption that the body part is located on the body part cushion can then be verified, embodying the function more robustly and reliably overall as a result. In the example of a head cushion, it can thus be verified whether a human head is actually located on the head cushion.

This, together with the magnetic field sensor and the corresponding position determination, results in more reliable detection of the body part position, so that the output of the magnetic resonance device does not have to be reduced unnecessarily, particularly with feet-first measurements or other measurements for which the patient is not lying with their head in a head coil. Even in the case of head-first measurements, it is not always confirmed that the patient's head is located in the designated head position. By means of the presence sensor, it is always possible to confirm beyond doubt whether the body part, particularly the head, is located on or in the body part cushion. Including after an SAR prediction or entirely independent thereof, the presence data can incidentally be evaluated advantageously, for example, so as to ascertain whether the body part remains consistently on the body part cushion throughout the measurement and/or to record the physical condition and stress level of the patient, which will be addressed in more detail below.

The body part can be any body part in principle, particularly a body part that has been laid on a pad for examination. The patient can be a living thing, particularly a human or an animal. The body part can particularly advantageously be a head. Determining position with the greatest possible accuracy can be particularly advantageous in the case of a head, because the head tends to be particularly prone to harm due to overheating, for example, also because of its relatively low mass (particularly compared to the generally much heavier torso). Alternatively, another body part can also be provided, however, such as a knee or elbow.

The term “body part cushion” refers generally to a cushion-like object that is suitable for supporting a body part. This body part cushion is preferably a head cushion. In particular, when a “body part cushion” is referred to in the context of this disclosure, the narrower term “head cushion” can be used instead as a variant, and when a “body part” is referred to, the narrower term “head” can be used instead as a variant. The body part of the patient is laid on the body part cushion. The body part cushion is preferably arranged on a patient table of the magnetic resonance device. In particular, the entire patient can be laid on the patient table, wherein the body part is laid indirectly over the body part cushion on the patient table. The body part cushion can additionally be used, in particular, to improve comfort and/or to support a specific positioning or orientation of the patient's body part. In a simple example, the patient can lie on their back (supine) and the body part, in this instance a head, lies on the body part cushion, in this instance a head cushion. Alternatively, the patient can also be laid on their stomach (prone). In this instance, it can be provided that the patient's face is facing the body part cushion or head cushion. The head cushion can comprise a recess, particularly a hole, for the face. Alternatively, the body part cushion can be a knee support for a knee, for example.

A body part cushion can commonly have a foam element on its upper side for comfortable support, for example. The foam element can, for example, extend over a base, which can be used for stabilization and/or connection to the patient table.

With the magnetic field sensor as part of the body part cushion, position data is determined using a magnetic field, particularly a stray field. The magnetic field sensor can, for example, be fixed externally (particularly externally on the side) to a base body of the body part cushion. The magnetic field sensor can be integrated at least partially in the base body of the body part cushion. A “magnetic stray field” can refer in particular to the magnetic field outside the patient opening (also: examination tunnel) or outside a homogeneity volume used for the imaging. The stray field can, in particular, be or comprise the stray field of the main magnetic field (B0 magnetic field) of the magnetic resonance device. While the magnetic field in the patient opening is largely homogeneous, this magnetic field decreases outside the patient opening, which can be provided as a hole in a main magnet unit. The magnetic field is usually smaller, the greater the distance from the patient opening. Information about the magnetic field distribution outside the patient opening can thus be used in connection with the measured stray field to infer the position of the magnetic field sensor and thus of the body part cushion, i.e., also of the body part. The position (and also the orientation if applicable) of the magnetic field sensor on the body part cushion is preferably known or defined in a fixed manner and is taken into account when determining position. A relative positioning of the body part on the body part cushion is preferably also known or specified and is taken into account when determining position. Optionally, the use of a plurality of magnetic field sensors on and/or in the body part cushion can be provided. A plurality of magnetic field sensors can, for example, be used to determine additional position information (for example, orientation). The position of the plurality of magnetic field sensors relative to the body part cushion, for example, relative to a geometric center of the body part cushion, can be defined in a fixed manner.

The position data can, for example, comprise the measured data of the magnetic field sensor. For example, the position data can be magnetic stray field measured data. The magnetic field sensor can be or comprise a three-dimensional magnetic field sensor, particularly a 3D Hall sensor. A three-dimensional magnetic field sensor is a magnetic field sensor that can record one magnetic field strength for each of three spatial directions. For example, three Hall elements can be provided that can each determine one magnetic field strength in one spatial direction. For example, the position data can comprise the magnetic field strength in three spatial directions. Optionally, the position data can already be at least partially processed by the magnetic field sensor and/or before it is transmitted to the evaluation device. For example, the measured data of the magnetic field sensor can be compressed, combined, or recalculated into another value.

The presence data and, if applicable, the position data are transmitted via a communication link. A means of transmission providing the communication link comprises the communication apparatus of the body part cushion, particularly comprising a transmitter, and can comprise a receiver that communicates with a control device of the magnetic resonance device.

According to one aspect, the presence data and, if applicable, the position data are transmitted wirelessly, particularly by radio transmission, and/or are transmitted via cable to the receiver. A cable transmission can, for example, be provided via an electrical connection using a coil cable. In the case of wireless transmission, the presence data and, if applicable, the position data can be transmitted from the communication apparatus, particularly comprising a transmitter, of the body part cushion to a receiver of the control device. In particular, in the case of wireless transmission, the body part cushion can have an energy storage device, which is designed to power the presence sensor, the magnetic field sensor, and/or the communication apparatus. The control device can also be designed to activate a safety mode in the event that no signals are received by the receiver. In safety mode, the magnetic resonance device can work in such a way that a maximum possible SAR is assumed for the body part, in particular, regardless of the actual position of the body part. This can be particularly advantageous with a wireless transmission, in order to be able to react to absent signals due to an empty energy storage device, for example. The advantage of cable transmission is that the body part cushion does not require an energy storage device. The magnetic field sensor and/or the presence sensor can be powered via the cabling, for example.

According to one aspect, the presence data and, if applicable, the position data are transmitted optically to the receiver, particularly using infrared light. Optical transmission represents a particularly advantageous possibility of wireless transmission. When the terms “optical” or “light” are used in the context of this disclosure, this generally means light in the broader sense, particularly also including infrared and UV light, unless otherwise specified. The use of infrared light is particularly preferable. While radio transmission often requires rather expensive radio approval, this approval is not usually required for an optical transmission.

Interference with other radio transmissions can also be ruled out. Moreover, it is advantageous that no cable is required, as this could otherwise be a distracting hindrance. Furthermore, optical transmission is advantageously also possible with relatively low energy expenditure, for example, using a light-emitting diode (LED). For example, the communication apparatus can comprise a light source or a light emitter, particularly an infrared light emitter (IR emitter). The IR emitter can comprise, in particular, an IR LED and an LED driver with a microcontroller. For example, the LED driver can be switched to ON or OFF using an IO pin of a microcontroller. The light emitter or the light source can be arranged and/or fixed at the edge of the body part cushion, for example. The light emitter or the light source is preferably placed in such a way on the body part cushion that a receiver, particularly an IR receiver, attached to a part of the magnetic resonance device, particularly the main magnet unit, can receive the optically transmitted presence data and, if applicable, position data.

According to one aspect, the presence data and, if applicable, the position data are transmitted by means of light pulses, particularly based on pulse-width modulation (PWM). For example, the microcontroller can also be designed to convert the presence data and, if applicable, the position data into a sequence of PWM pulses. The sequence of PWM pulses can be emitted accordingly from the IR LED. The sequence of PWM pulses can follow a defined protocol (for example, IR link or TOSLINK). A particularly simple and, at the same time, reliable transmission can advantageously be facilitated by means of light pulses.

In one particularly advantageous development of the disclosure, it can be provided that at least one of the at least one presence sensors is a temperature sensor, measuring infrared radiation in particular from an object positioned on the body part cushion. By means of one or more temperature sensors in the body part cushion, it can be established with high certainty whether something warm/living is located on the body part cushion. A control device of the magnetic resonance device evaluating the presence data can, for example, verify whether the temperature measured by the temperature sensor, corresponding to a temperature of the object, lies within an accepted temperature range for the patient, particularly a human, for example, around 37° C. It can thus be confirmed, particularly when using additional presence sensors with other measuring principles, that a random object has not been placed on the body part cushion, which could mislead the equipment, for example, an SAR monitor.

The temperature sensor can, in particular, be an infrared sensor. For example, an infrared sensor measuring in the far infrared range, particularly around 10 μm, can be used. It is particularly expedient if the temperature sensor is aimed at the neck or ears of the patient and/or is foamed in a foam element of the body part cushion. For example, the temperature sensor can therefore be aimed at a specific point on the patient, at the ears or neck with respect to the head. This avoids taking measurements in the area of the hair of the patient, as the temperature cannot be detected beyond doubt.

In particular, the temperature sensor can be a thermal MEMS presence sensor. Such sensors can detect the slightest change in temperature, which can be used in a wide variety of applications. The high sensitivity facilitates the detection of the stationary human presence. A high signal-to-noise ratio can be achieved by using MEMS and, in particular, ASIC technology.

It is particularly expedient to combine the temperature sensor as a presence sensor with other presence sensors, in order to collate information with various measuring principles and thus be able to detect the presence of the body part on the body part cushion more robustly and reliably.

In order to achieve the greatest possible measuring accuracy, while also allowing other functions, the temperature sensor can also be provided with an optical arrangement for alignment with the specific point and/or to boost the infrared radiation coming from there, and/or a recess can be provided in the body part cushion from the temperature sensor to the surface, on which the specific point is placed. It is also conceivable to arrange the temperature sensor at the edge of the foam element, particularly on the surface, or next to this, behind a cover material, for example, and/or to arrange the temperature sensor in such a way laterally to the body part cushion that it at least partially continues its surface on which the body part is intended to be laid. Applications in which even physiological values, such as a pulse, were acquired by means of an infrared sensor, have already been described in relation to mobile devices, for example, cell phones, and can also be used within the scope of the present disclosure, in order to evaluate the presence data.

In an expedient, preferred development of the disclosure, it can be provided that at least one of the at least one presence sensors is a pressure sensor. One or more pressure sensors can be used to verify, in particular, whether a specific weight is on the body part cushion. In the example of a head cushion, it is known that the weight of a human head is in the range from 3 to 4 kg. Thus, presence data from one or more pressure sensors can be used to verify whether the weight on the body part cushion is within a specified permissible weight range. The permissible weight range can also be determined on a patient-specific basis, for example, if information is taken into account that was obtained when the patient was registered. By verifying in this way, it is possible to determine whether the correct body part, for example, a head, is actually present on the body part cushion, or whether another body part, for example, a foot, another object, for example, a phantom, or even nothing at all is present instead. For example, a load cell can be used as a pressure sensor, and/or the pressure sensor can be based on a strain gage. The electrical resistance in such pressure sensors changes when force is exerted. An optical pressure sensor can also be used as a pressure sensor, for example, a fiber Bragg grating sensor.

In the present application for the at least one pressure sensor, it must be taken into account that a body part cushion has to be soft, so the pressure of the body part distributes unevenly into the body part cushion, for example, the foam element. In order to avoid falsification of the presence data due to this pressure distribution, a particularly advantageous development of the present disclosure provides that the body part cushion has two parallel base plates arranged at different heights in a lower area of the body part cushion, of which the upper plate is movable against the lower and between which the multiple pressure sensors are arranged. In other words, two fixed base plates, which are made from PVC, for example, are provided in the lower part of the body part cushion, arranged one above the other. The pressure sensors are arranged between these two base plates. A particularly advantageous arrangement arises if at least five pressure sensors are present, of which one is arranged in the center and four at the corners of the upper plate. Known methods can then be used to determine the weight that has caused the pressure based on the presence data from the multiple pressure sensors.

In one aspect of the disclosure, it can further be provided that at least one of the at least one presence sensors is a strain gage integrated in a or the foam element. In particular, such a strain gage can be used as an alternative solution to the at least one pressure sensor. If a strain gage is integrated in the body part cushion, in particular is foamed there, a change in resistance can be detected when this is stretched. When such a change in resistance occurs, an object has been placed on the body part cushion. A weight can also be estimated here. As also applies to the aspect with the pressure sensor, a combination with the temperature sensor can be used to detect beyond doubt that this is a human body part.

In aspects of the present disclosure, it is also conceivable that at least one of the at least one presence sensors is a capacitive sensor. A capacitive sensor can also be used in particular as an alternative to a pressure sensor and/or a strain gage. Once a change is detected, here too, an object has been placed on or in the body part cushion. In advantageous developments, the capacitive sensor can be aligned at least primarily according to an expected progression of the surface of the body part, in order to optimize measuring characteristics and thus measuring accuracy. It can, in particular, be foamed here in a foam element.

Overall, the use of a pressure sensor represents the preferred variant in comparison to a capacitive sensor and/or a strain gage. A pressure sensor can be operated with extremely low electrical power and uses direct current, thus no radio frequency, making it particularly compatible for magnetic resonance measurements. Additionally, it can be arranged away from the body part, particularly when arranged between base plates, which is also beneficial for compatibility with magnetic resonance imaging.

Thus, it is particularly preferable to use one temperature sensor and at least one pressure sensor and/or at least one strain gage or at least one capacitive sensor. The temperature determination can be used to establish whether this is a person or whether another object has been placed on the body part cushion. The weight measurement or the capacitive measurement can be used to determine whether the correct body part is located on the body part cushion rather than another (warm) body part, such as a foot on a head cushion.

With respect to the magnetic resonance compatibility of the used presence sensors, it should be noted that these can have at least one means of shielding and/or are arranged far enough away from the imaging area to be scanned in any case, so there is no interference with the imaging operation.

In addition to the body part cushion, the present disclosure also relates to a magnetic resonance device, having a body part cushion according to the disclosure, the receiver for presence data from the body part cushion, and a control device connected to the receiver for evaluating the presence data, wherein the control device is designed to use the presence data for determining presence information describing the presence of the associated body part on the body part cushion. All statements concerning the body part cushion according to the disclosure can also be applied analogously to the magnetic resonance device according to the disclosure and vice versa, so that the aforementioned advantages can also be achieved with the magnetic resonance device.

As already explained, one particularly advantageous area of application arises if the body part cushion also has a magnetic field sensor for acquiring the local magnetic field as position data and the communication apparatus is also designed to transmit the position data to the receiver, wherein the control device is designed to use the presence information for determining a trigger for the acquisition of position data and/or for predicting a specific absorption rate in an evaluation using the position data. In particular, it can thus be provided that the position is only determined by means of the position data from the magnetic field sensor if the presence information actually also indicates the presence of the body part. Verification that the presence information indicates the presence of the body part can, for example, act as a trigger for acquiring the position data and also transmitting this to the receiver. An SAR can then be predicted there in particular, as described in the already referenced subsequently published application.

When using at least one temperature sensor, the control device can be designed to determine a temperature of an object placed on the body part cushion and to compare this to a permissible temperature range for the associated body part, when determining the presence information from its presence data. For a human, the permissible temperature range can, for example, be around 37° C., particularly from 35° C. to 39° C. If it is not possible to measure the temperature of the body part directly due to the arrangement of the temperature sensor, for example, or there is systematic deviation, the control device can take this into account accordingly.

In the preferred exemplary aspects, it can be provided that the control device is designed, when using at least one pressure sensor, to determine a weight of an object placed on the body part cushion and to compare this to a permissible weight range for the associated body part, when determining the presence information from its presence data. As already explained, the permissible weight range for the body part can be predefined in a fixed manner here and/or can be determined on a patient-specific basis using patient information, particularly patient height and/or patient weight. The presence of the body part can be verified with greater reliability in this way. For a head, for example, the permissible weight range can be between 3 and 7 kg.

As already mentioned, the presence data can also be otherwise used and evaluated in addition to or as an alternative to use in respect of an SAR prediction. For example, it is conceivable not to start a magnetic resonance measurement until the body part is located (in particular consistently throughout a specified waiting period) on the body part cushion, as this indicates whether the patient is correctly positioned and/or is calm.

Furthermore, a development of the disclosure can also provide that the control device is designed to evaluate presence data that is available at multiple points during a period of time for determining change information, in particular indicating a movement of the body part. In other words, it is possible to verify whether the patient's body part remains consistently on the body part cushion throughout the measurement or whether a movement occurs that can be detected by comparing the presence data. For example, there is movement if the temperature and/or the pressure change rapidly. Depending on the respective situation, for example, whether the corresponding body part has been scanned, canceling and/or interrupting the measurement can be considered. It can be particularly advantageous to at least log the change information indicating that movement has occurred, for example, as DICOM metadata and/or in a log file, which can be allocated to the imaging result.

In another aspect, it can also be provided that at least one of the at least one presence sensors is a temperature sensor and the control device is designed to determine state information from the presence data of the temperature sensor, which describes the physiological and/or psychological state of the patient. For example, the temperature measurement can be used to determine the physical condition and/or stress level of the patient. This is particularly expedient if an infrared sensor that measures with sufficient accuracy is being used, but it can also be achieved with other types of temperature sensors. Corresponding evaluation methods are already known from other areas of application.

In one specific development, it can then be provided that the change information and/or the patient information are used by the control device when controlling the magnetic resonance device during an examination process, particularly for canceling and/or interrupting a measurement when a cancellation condition is met by change information and/or state information, wherein the cancellation condition describes an intensity of change in excess of a change threshold and/or describes a patient state that falls short of a threshold for measuring patient state. Thus, the presence data can be used to facilitate both high-quality measurement and patient protection.

Finally, the disclosure also relates to a method for determining the presence of a body part, particularly a head, of a patient in a magnetic resonance device, wherein the method comprises the following steps:
provision of a body part cushion, wherein the body part cushion is arranged in particular on a patient table of the magnetic resonance device,
acquisition of presence data with a presence sensor, which is arranged in or on the body part cushion,
transmission of the presence data to a control device via a communication link between a communication apparatus of the body part cushion and a receiver,
determination of presence information describing the presence of the associated body part on the body part cushion by the control device from the presence data.

The statements concerning the body part cushion according to the disclosure and the magnetic resonance device according to the disclosure can also be applied to the method according to the disclosure, so that the stated advantages are also applicable here. The method, according to the disclosure, can be understood as a method for determining presence.
In particular, a method can thereby also arise for determining a specific absorption rate of a body part, particularly a head, of a patient in a magnetic resonance device, i.e., an SAR determination method, comprising the following steps: execution of a presence determination method according to the disclosure, in order to determine presence information for the body part; in the case of presence information indicating the presence of the body part, determination of a magnetic field, particularly a stray field, with a magnetic field sensor, which is arranged in or on the body part cushion, in order to calculate position data, wherein the magnetic field sensor comprises in particular a 3D Hall sensor; transmission of the position data to the control device; determination by the control device of the position of the body part in the magnetic resonance device using the position data based on the fact that the body part is laid on the body part cushion; and determination of the specific absorption rate of the body part based on an expected field distribution during a measurement of the magnetic resonance device and the determined position of the body part.

Once the presence of the body part has been verified, an SAR can be estimated with greater reliability, accuracy, and robustness.

### BRIEF DESCRIPTION OF THE DRAWINGS

Further advantages and details of the present disclosure are specified in the exemplary aspects described below and also with reference to the drawings, in which:

FIG. 1 shows a schematic diagram of a magnetic resonance device according to the disclosure.

FIG. 2 shows a schematic cross-sectional side view of a first exemplary aspect of a body part cushion according to the disclosure.

FIG. 3 shows a schematic cross-sectional view of the first exemplary aspect from behind,

FIG. 4 shows a schematic representation of the arrangement of a strain gage in a second exemplary aspect of the body part cushion according to the disclosure.

FIG. 5 shows a schematic top view of the arrangement of a capacitive sensor in a third exemplary aspect of the body part cushion according to the disclosure.

FIG. 6 shows a schematic side view of the arrangement in FIG. 5.

FIG. 7 shows a flowchart of an exemplary aspect of the method according to the disclosure.

### DETAILED DESCRIPTION

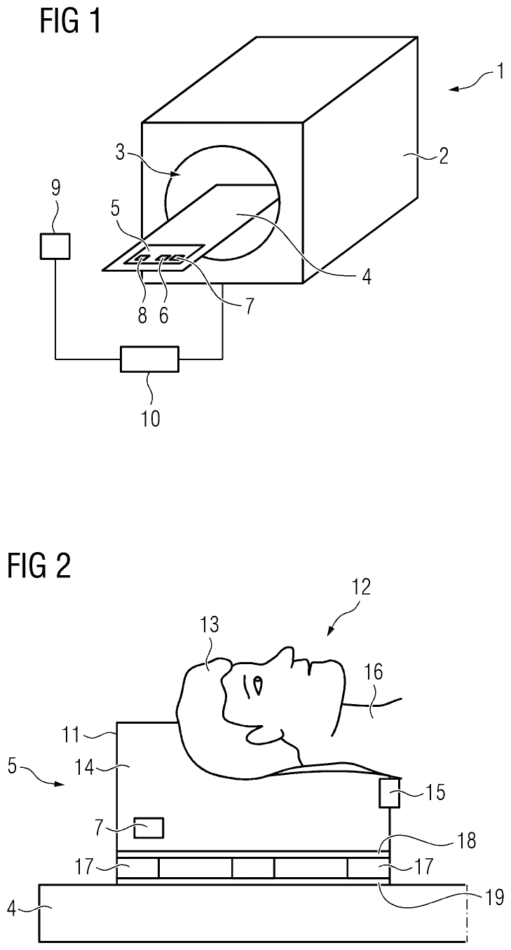

FIG. 1 shows a schematic diagram of an exemplary aspect of a magnetic resonance device 1 according to the disclosure. The magnetic resonance device 1 comprises a main magnet unit 2, which comprises the in particular superconducting main magnet not shown in greater detail and has a cylindrical patient opening 3, into which a patient can be moved by means of a patient table 4 for a magnetic resonance examination. A high-frequency coil arrangement, not shown in greater detail, and a gradient coil arrangement, not shown in greater detail, can be provided enclosing the patient opening 3. Local coil arrangements can also be provided, particularly on the patient table 4.

A body part cushion 5 according to the disclosure, on which a specific body part of the patient is to be placed, is arranged on the patient table 4. The body part cushion 5 comprises at least one presence sensor 6 and at least one magnetic field sensor 7. Presence data from the presence sensor 6 and position data from the magnetic field sensor 7, which is incidentally embodied as a three-dimensional Hall sensor, can be transmitted by means of a communication apparatus 8 to a receiver 9, which is connected to a control device 10, which controls the operation of the magnetic resonance device 1 and is designed to evaluate the presence data and the position data. The communication apparatus 8 and the receiver 9 form a means of transmission for the presence data and the position data.

While the presence data and the position data can be transmitted by cable in principle, a wireless communication link is provided in the present disclosure. The presence data and the position data are preferably transmitted optically here, which is why two receivers 9 can be provided, for example, each of which can be arranged in a shielded room, in which the main magnet unit 2 is arranged, in a longitudinal direction in front of or behind the patient opening 3.

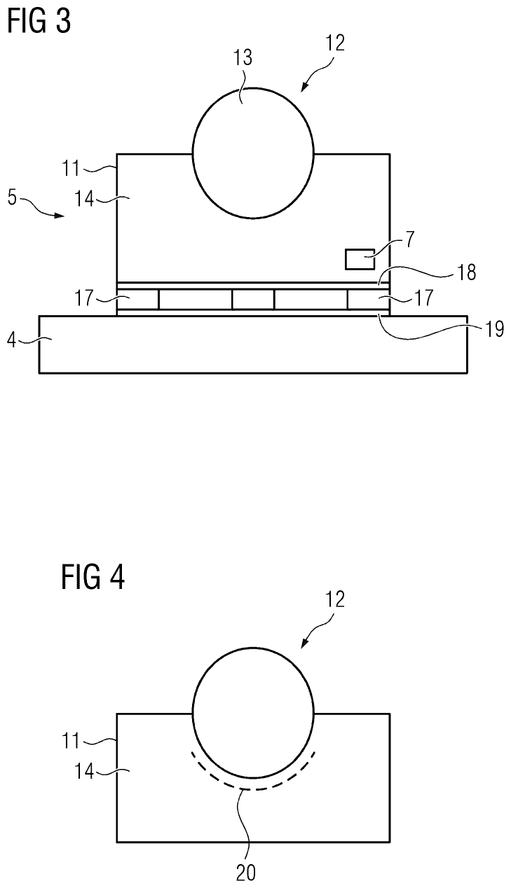

FIGS. 2 and 3 show a specific first exemplary aspect of a body part cushion 5, which is embodied here as a head cushion 11. A head 12 of the patient, with hair 13, is shown schematically as the body part that is arranged on the body part cushion 5. The head cushion 11 is clearly arranged on the patient table 4.

In addition to the foamed magnetic field sensor 7, here within a foam element 14 of the head cushion 11, the head cushion 11 has multiple presence sensors 6. The communication apparatus 8 is not shown for reasons of clarity.

A temperature sensor 15 is provided as a first presence sensor 6, which is embodied here as an infrared sensor, and is arranged at the edge of the foam element 14 in such a way that it is aimed at the neck 16 of the patient. Other orientations are also conceivable, provided that the hair 13 does not prevent a reliable measurement. The temperature sensor 15 can alternatively also be foamed in the foam element 14.

The body part cushion 5 comprises multiple, here five, pressure sensors 17 as additional presence sensors 6, which are arranged between an upper base plate 18 and a lower base plate 19, which can be made from PVC, for example, and are movable relative to each other. As is also evident from the views in FIG. 2 and FIG. 3, a pressure sensor 17 is arranged here at each corner of the area between the base plates 18, 19 and in the center. The pressure sensors 17 are embodied as load cells by way of example. The electrical resistance changes in these when pressure is exerted, so that a weight laid on the head cushion 11 can be inferred from the presence data in particular. The fixed base plates 18, 19 prevent the pressure distribution of the head 12 on the foam element 14 falsifying the measurement.

The provision of both the temperature sensor 15 and the pressure sensors 17 allow for the presence of the correct body part on the body part cushion to be determined in a particularly more robust and reliable manner. The temperature measurement can be used to establish whether this is a person or whether another object, for example, a phantom, has been laid there. The weight measurement from the pressure sensors 17 (or another measurement still to be discussed below) can be used to determine whether the head 12 is located on the head cushion 11 rather than another (warm) body part, such as a foot.

FIGS. 4, 5, and 6 schematically illustrate other conceivable aspects of presence sensors 6, in order to supplement the temperature sensor 15, for example. FIG. 4 shows a schematic representation of a foamed strain gage 20 here as a presence sensor 6 in the foam element 14 of the head cushion 11. The strain gage 20 can, for example, cover 30 to 60% of the width of the foam element 14 and be arranged near the contact surface for the head 12 and in parallel to this. As is apparent from FIG. 4, when the head 12 is positioned on the head cushion 11, the form of the strain gage 20 changes by stretching, so that the resistance changes, from which the strength of the deformation and thus the effective weight force can be deduced.

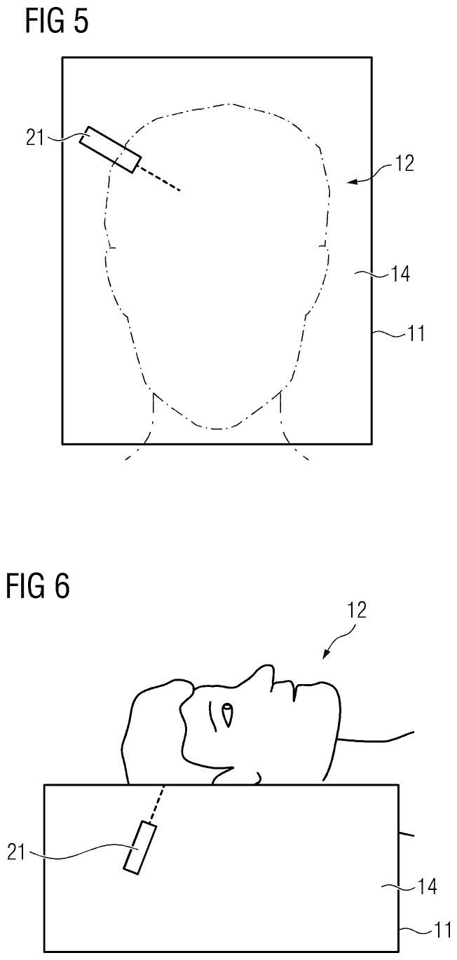

FIGS. 5 and 6 also schematically show a foamed capacitive sensor 21 as a presence sensor 6 in the foam element 14. The capacitive sensor 21 is arranged in the foam element 14 so that its coverage corresponds to the expected progression of the head 12.

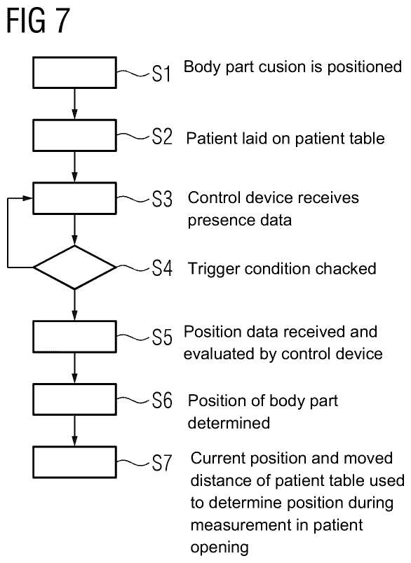

FIG. 7 shows a flowchart of a method according to the disclosure. According to this, the body part cushion 5, as a head cushion 11, for example, is first positioned in a step S1 at the foot of the patient table 4 and connected to the control device 10 in a wired or wireless manner. The patient is then laid on the patient table 4 in a step S2.

The control device 10 thereby receives presence data from the presence sensors 6 in step S3 and evaluates this to determine presence information. The presence data from the temperature sensor 15 is used here to verify whether the measured temperature is within a permissible range for a human patient, for example, from 35° C. to 39° C. The presence data from the weight sensors 17 is used to verify whether the weight placed on the body part cushion 5 and detected by these sensors is within a permissible weight range for the body part, in the example, the head 12. For example, the permissible weight range for the head 12 can be specified as from 3 kg to 4 kg; however, the permissible weight range is preferably determined on a patient-specific basis. Patient information acquired during registration of the patient or by other means, such as patient height and patient weight, can also be evaluated, in order to determine a specifically expected permissible weight range for the head 12 of this patient. If a strain gage 20 and/or a capacitive sensor 21 are provided instead of or in addition to the pressure sensors 17, corresponding or analogous evaluation methods can be used.

If the temperature is within the permissible temperature range and the weight is within the permissible weight range, the presence information indicates that the correct body part is on the body part cushion 5, otherwise that this is not the case.

A trigger condition is checked in a step S4 to verify whether the presence information indicates the presence of the body part on the body part cushion 5. If this trigger condition is met, a magnetic field, here a stray field of the main magnetic field, is measured with the magnetic field sensor 7 in a step S5, in order to acquire position data that is transmitted to the control device 10.

The position data is received and evaluated by the control device 10 in a step S5, in order to determine the position of the body part in the magnetic resonance device 1 using the position data based on the fact that the body part is lying on the body part cushion 5.

Thus, the position data is used to determine the position of the body part in a coordinate system of the magnetic resonance device 1. For example, it can be provided that a position along the longitudinal axis of the patient opening 3 (z-position) is determined from a value of the measured stray field and/or a relative position relative to an isocenter of the main magnet (center of the homogeneity volume). Once the position of the patient table 4 is known in the coordinate system of the magnetic resonance device 1 and is tracked when the patient table 4 is moved, a position of the body part can be determined at a specific later point in time from a travel range of the patient table 4, which also applies to predicting its position, for example, for an SAR prediction with respect to a measurement of the magnetic resonance examination. The aforementioned patient information, particularly the height or length information for the patient, can incidentally also be used to determine position.

In particular, here the patient is first (step S2) placed on the patient table 4 and the body part cushion 5 outside the patient opening 3, and the position of the body part is determined (steps S5 and S6) with the magnetic stray field (present there).

Since it is now known how the patient table 4 has subsequently moved with the body part in the patient opening 3, which relates to the moved distance, in particular, the current position and the moved distance of the patient table 4 can then be used in a step S7 to determine the position during the measurement in the patient opening 3. This is now used to predict the specific absorption rate (SAR) of the body part, particularly of the head 12, based on an expected field distribution during the measurement of the magnetic resonance device 1.

If the control device 10 acquires no presence data and/or position data, a safety mode can be activated, in which a worst-case scenario is assumed with respect to the position of the head.

It should be noted that the presence data can still be acquired and sent to the control device 10 to be evaluated there, even after the completion of step S7 (in which, if applicable, a magnetic resonance sequence or scanning protocol to be used can at least be adjusted, in order to comply with SAR limit values). In particular, the presence data facilitates monitoring of whether the patient is keeping the body part consistently on the body part cushion 5, particularly throughout the measurement. If the temperature or pressure suddenly changes sharply, a movement has occurred, which can be described by change information that can be determined by the control device 10. Moreover, it is also possible to use the temperature measurement to assess the patient's physiological and psychological state, which is why state information can be determined.

Regardless of the change information and/or the state information, the control device 10 can control or influence the operation of the magnetic resonance device 1, for example, in an extreme case for canceling and/or interrupting a measurement when a cancellation condition is met by change information and/or state information, wherein the cancellation condition describes an intensity of change in excess of a change threshold and/or describes a patient state that falls short of a threshold for measuring patient state. Thus, the change information can, for example, comprise a measure for assessing the intensity of change, and/or the state information can comprise a measure that describes the extent to which the patient's state is good or bad. In particular, the use of multiple measures and accordingly of multiple thresholds can be provided, depending on the measuring principle and/or aspect of state.

Independent of the grammatical term usage, individuals with male, female, or other gender identities are included within the term.

1. A body part cushion for supporting a body part during a magnetic resonance examination with a magnetic resonance device, comprising:
at least one presence sensor configured to acquire presence data related to the presence of the body part; and
a communication apparatus configured to transmit the presence data to a receiver.

2. The body part cushion as claimed in claim 1, further comprising:
a magnetic field sensor configured to acquire a local magnetic field as position data,
wherein the communication apparatus is also configured to transmit the position data to the receiver.

3. The body part cushion as claimed in claim 1, wherein at least one of the at least one presence sensor is a temperature sensor positioned on the body part cushion.

4. The body part cushion as claimed in claim 3, wherein the temperature sensor is aimed at a neck or ears of a patient and/or is foamed in a foam element of the body part cushion.

5. The body part cushion as claimed in claim 1, wherein at least one of the at least one presence sensor is a pressure sensor.

6. The body part cushion as claimed in claim 5, further comprising:
upper and lower parallel base plates arranged at different heights in a lower area of the body part cushion, of which the upper plate is movable against the lower base plate, and between which multiple pressure sensors are arranged.

7. The body part cushion as claimed in claim 6, further comprising:
at least five pressure sensors arranged between the base plate, with one arranged in a center and four at corners of the upper and lower parallel base plates.

8. The body part cushion as claimed in claim 1, wherein at least one of the at least one presence sensors is a strain gage integrated in a foam element of the body part cushion.

9. The body part cushion as claimed in claim 1, wherein at least one of the at least one presence sensors is a capacitive sensor.

10. The body part cushion as claimed in claim 9, wherein the capacitive sensor is aligned at least primarily according to an expected progression of q surface of the body part and/or is foamed in a foam element of the body part cushion.

11. A magnetic resonance device, comprising:
a body part cushion as claimed in claim 1;
a receiver configured to receive presence data from the body part cushion; and
a control device connected to the receiver and configured to evaluate the presence data, wherein the control device is configured to use the presence data to determine presence information indicating the presence of the associated body part on the body part cushion.

12. The magnetic resonance device as claimed in claim 11, wherein the body part cushion also comprises a magnetic field sensor configured to acquire a local magnetic field as position data and the communication apparatus is also configured to transmit the position data to the receiver, wherein the control device is configured to use the presence information to determine a trigger for the acquisition of position data and/or to predict a specific absorption rate in an evaluation using the position data.

13. The magnetic resonance device as claimed in claim 11, wherein the control device is configured to determine a weight of an object placed on the body part cushion and to compare this to a permissible weight range for the associated body part, when using at least one pressure sensor when determining the presence information from its presence data.

14. The magnetic resonance device as claimed in claim 11, wherein the control device is configured to evaluate presence data that is available at multiple points during a period of time to determine change information, and/or at least one of the at least one presence sensors is a temperature sensor and the control device is configured to determine state information from the presence data of the temperature sensor, which describes a physiological and/or psychological state of a patient.

15. A method for determining a presence of a body part of a patient in a magnetic resonance device, wherein the method comprises:
providing a body part cushion, which is arranged in the magnetic resonance device;
acquiring presence data with a presence sensor, which is arranged in or on the body part cushion,
transmitting the presence data to a control device via a communication link between a communication apparatus of the body part cushion and a receiver; and
determining presence information describing the presence of the associated body part on the body part cushion by the control device from the presence data.
